(19) United States
(12) Patent Application Publication
(10) Pub. No.: US 2022/0084678 A1
(43) Pub. Date: Mar. 17, 2022

(54) AI-BASED DETECTION OF PROCESS ANOMALIES IN USAGE DATA FROM PATIENT EXAMINATION DEVICES IN HEALTHCARE

(71) Applicant: Siemens Healthcare GmbH, Erlangen (DE)

(72) Inventors: Michael KELM, Erlangen (DE); Alexander RICHTER, Erlangen (DE); Jan SCHREIBER, Muenchen (DE)

(73) Assignee: Siemens Healthcare GmbH, Erlangen (DE)

(21) Appl. No.: 17/462,156

(22) Filed: Aug. 31, 2021

(30) Foreign Application Priority Data
Sep. 11, 2020 (DE) 10 2020 211 459.5

(51) Int. Cl. G16H 50/20; G06N 20/00; G16H 50/30

(52) U.S. Cl. CPC G16H 50/20; G16H 50/30; G06N 20/00

(57) ABSTRACT

A computer-implemented methods is for training a machine learning algorithm to detect anomalies in processes. A computer-implemented method is for detecting anomalies in processes. Further, data processing systems and a system are for detecting anomalies in processes. A training dataset of a process is provided including a first number of training usage sequences. A second number of process trees is created based upon the usage sequences by way of a process mining algorithm. The creation of the process trees is subject to a certain randomness.

### PRIORITY STATEMENT

The present application hereby claims priority under 35 U.S.C. § 119 to German patent application number DE 102020211459.5 filed Sep. 11, 2020, the entire contents of which are hereby incorporated herein by reference.

### FIELD

Example embodiments of the invention generally relate to computer-implemented methods for training a machine learning algorithm to detect anomalies in processes; to a computer-implemented method for detecting anomalies in processes; and to corresponding data processing systems and also to a system for detecting anomalies in processes.

### BACKGROUND

Processes or workflows including several different activities are performed in the most diverse sectors and disciplines in order to achieve desired predefined results. In a manufacturing process, for instance, specific activities such as manufacturing steps, settings for process control variables, etc. can be performed sequentially or simultaneously in order to produce a predefined product from one or more base materials or raw materials. It is also possible in an application process to perform specific activities such as actions, settings for application variables, etc. sequentially or simultaneously in order to achieve a predefined application result on a subject (e.g. product, patient, etc.). In processes that are performed using, or at, machines or devices, it is usual to collect large amounts of data, in particular log-data, that logs the usage sequences carried out. A standardized process containing a standardized sequence of always identical process steps is generally deemed advantageous here.

Modern healthcare and in particular clinical diagnosis and treatment rely heavily on highly developed technical examination devices such as diagnostic imaging devices (for example ultrasound devices, X-ray devices, computed tomography devices, magnetic resonance tomography devices (MRT), positron emission tomography devices (PET), digital pathology scanners, genome-sequencing devices/microarrays) and various automated laboratory devices that measure health-related values of biological samples. In addition, therapeutic devices such as robot-assisted surgical systems, semi-automatic or fully automatic implantation instruments, etc. are used for treatment by following predefined work steps. Many of these examination and therapeutic devices require significant investment both in the device itself and in staff training. Health service providers are therefore interested in optimizing the use of such devices as far as possible.

With the emergence of cloud technologies and the trend towards the Medical Internet of Things (MIoT), such devices are providing ever more metadata (for instance log data/logs), which can be used to monitor an implementation of an examination, or in other words monitor a process, in detail. This means that different types of examinations requiring different and varying preparatory and measurement steps can be observed along with timings, possible error conditions or warning conditions and sensor measurements. If a large number of such devices are being operated, the health providers are interested in maintaining a “normal” operation of patient examinations, and finding out about “abnormal” patient examinations having negative impacts on quality or efficiency, in order to be able to rectify as quickly as possible (systematic) incorrect protocols or processes.

Radiological MRT examinations are a possible example here. In a daily routine, patient examinations are carried out on different MRT scanners using different types of procedures. Every procedure consists of a series of protocol steps that must be carried out for a specific type of examination. When performing such procedures, medical personnel are meant to follow guideline protocol sequences, also known as guidelines. The guideline protocol sequences specify what protocol steps must be carried out in what order. This prevents missing clinical findings or harm to a patient. In addition, it is easier to interpret scans, and the scan duration can be standardized, which is essential to cost effectiveness and efficient planning of examinations. Observed usage sequences in clinical use sometimes deviate from an expected guideline protocol sequence. The deviation may be due to medical circumstances requiring an ad hoc decision or may have been caused by incorrect operation of the device. Medical experts are interested in systematic deviations, i.e. anomalies, because the systematic deviations can indicate, for example, errors or inefficiencies in defined guideline protocol sequences, or the need for additional training of medical personnel. Manual analysis of logged usage sequences often is not feasible or is impractical because of a large number of examinations.

Formally, the above problem results in a collection of logged usage sequences that can be modeled as observations of implementations of a process.

### SUMMARY

At least one embodiment of the present invention determines anomalies in processes in an automated manner. To this end, at least one embodiment of the present invention provides computer-implemented methods for training a machine learning algorithm to detect anomalies in processes. Embodiments of the present invention are directed to a corresponding data processing system, a computer-implemented method, a data processing system and a system for detecting anomalies in processes. The subject matter of the respective claims contains developments and embodiments of the present invention.

According to a first embodiment of the present invention, a computer-implemented method is for training a machine learning algorithm to detect anomalies in processes comprising:

providing a training dataset of a process, comprising a first number N of training usage sequences, wherein the first number N is greater than one (N>1);

creating a predefined second number K of bootstrap datasets, wherein the second number K is greater than one (K>1), based upon the training dataset by randomly drawing a predefined third number M of usage sequences from the training dataset, wherein each draw is made from the first number N of training usage sequences; and

creating a number of process trees equal to the second number K by creating each process tree based upon one of the bootstrap datasets by way of a process mining algorithm.

According to a second embodiment of the present invention, a computer-implemented method for training a machine learning algorithm to detect anomalies in processes comprises:

providing a training dataset of a process, comprising a first number N of training usage sequences, wherein the first number N is greater than one; and

creating a second number K of process trees, wherein the second number K is greater than one (K>1), by creating each process tree based upon the training dataset by way of a process mining algorithm, wherein in each operator node of each process tree, one of the possible split operators is randomly selected.

According to a third embodiment of the present invention, a data processing system comprises at least one processor or electronic circuit for performing the method according to the first or second embodiment of the present invention.

According to a fourth embodiment of the present invention, a computer program product comprises commands which, on execution of the computer program product by a computer, cause the computer to perform the method according to the first or second embodiment of the present invention.

According to a fifth embodiment of the present invention, the computer program product according to the fourth embodiment of the present invention is stored on a computer-readable data storage medium.

According to a sixth embodiment of the present invention, a computer-implemented method for detecting anomalies in processes, in particular anomalies in processes by medical devices, comprises:

receiving a usage sequence of a process.

determining a prediction vector based upon the received usage sequence by way of a trained machine learning algorithm that has been trained by the method according to the first or second embodiment of the present invention, wherein the prediction vector comprises for each process tree of the trained MLA a value indicating whether the usage sequence fits the process tree concerned;

determining a normalized fitness value from the prediction vector and the second number K; and

classifying the process based upon the normalized fitness value.

According to a seventh embodiment of the present invention, a data processing system comprises at least one processor or electronic circuit for performing the method according to the sixth embodiment of the present invention.

According to an eighth embodiment of the present invention, a computer program product comprises commands which, on execution of the computer program product by a computer, cause the computer to perform the method according to the sixth embodiment of the present invention.

According to a ninth embodiment of the present invention, the computer program product according to the eighth embodiment of the present invention is stored on a computer-readable data storage medium.

According to a tenth embodiment of the present invention, a system for detecting anomalies in processes comprises a medical device and the data processing system according to the ninth embodiment of the present invention. The medical device is designed to log usage sequences relating to processes. The data processing system is communicatively connected to the medical device and designed to receive the usage sequences from the medical device.

According to another embodiment of the present invention, a computer-implemented method is for training a machine learning algorithm to detect at least one anomaly in at least one process, comprising:

providing a training dataset of a process, including a first number of training usage sequences, the first number being greater than one;

creating a second number of bootstrap datasets, the second number being greater than one, based upon the training dataset by randomly drawing a third number of usage sequences from the training dataset, each draw of the drawing of the third number being made from the first number of training usage sequences; and

creating a number of process trees equal to the second number by creating each process tree, of the number of process trees, based upon one bootstrap dataset of the bootstrap datasets, using a process mining algorithm.

According to another embodiment of the present invention, a computer-implemented method is for training a machine learning algorithm to detect at least one anomaly in at least one process, comprising:

providing a training dataset of a process, including a first number of training usage sequences, the first number being greater than one; and

creating a second number of process trees, the second number being greater than one, by creating each process tree, of the number of process trees, based upon the training dataset using a process mining algorithm, wherein in each operator node of each process tree, one of a possible split of operators is randomly selected.

According to another embodiment of the present invention, a computer-implemented method is for training a machine learning algorithm to detect at least one anomaly in at least one process, comprising:

providing a training dataset of a process, including a first number of training usage sequences, the first number being greater than one;

creating a second number of bootstrap datasets, the second number being greater than one, based upon the training dataset by randomly drawing a third number of usage sequences from the training dataset, wherein each draw of the drawing of the third number is made from the first number of training usage sequences; and

creating a number of process trees equal to the second number by creating each process tree, of the number of process trees, based upon one of the bootstrap datasets using a process mining algorithm, wherein in each operator node of each process tree, one of a possible split of operators is randomly selected.

According to another embodiment of the present invention, a data processing system comprises at least one processor or electronic circuit for performing at least the computer-implemented method of an embodiment.

According to another embodiment of the present invention, a computer-implemented method is for detecting at least one anomaly in at least one process, comprising:

receiving a usage sequence of a process;

determining a prediction vector based upon the usage sequence received by use of a trained machine learning algorithm trained by the computer-implemented method of an embodiment, wherein the prediction vector includes, for each respective process tree of the trained machine learning algorithm, a value indicating whether the usage sequence fits the respective process tree;

determining a normalized fitness value from the prediction vector and the second number; and

classifying the process based upon the normalized fitness value.

According to another embodiment of the present invention, a system is for detecting at least one anomaly in at least one process, comprising:

a medical device, embodied to log usage sequences relating to processes; and

the data processing system of an embodiment, the data processing system being communicatively connected to the medical device and being embodied to receive the usage sequences from the medical device.

### BRIEF DESCRIPTION OF THE DRAWINGS

The following description of the example embodiments will clarify and elucidate the above-described properties, features and advantages of this invention, and the manner in which they are achieved, which example embodiments are explained in greater detail in conjunction with the drawings,

in which:

FIGS. 1-3 show example embodiments of the method according to the first embodiment of the present invention and according to the second embodiment of the present invention;

FIG. 4 shows another representation of the method of FIG. 3;

FIG. 5 is a schematic representation of a directly-follows graph;

FIG. 6 is a schematic representation of a process tree;

FIG. 7 shows an example embodiment of the data processing system according to the third embodiment of the present invention;

FIG. 8 shows an example embodiment of the computer-readable data storage medium according to the fifth embodiment of the present invention;

FIG. 9 shows an example embodiment of the method according to the sixth embodiment of the present invention;

FIG. 10 shows another representation of the method of FIG. 9;

FIG. 11 shows an example embodiment of the data processing system according to the seventh embodiment of the present invention;

FIG. 12 shows an example embodiment of the computer-readable data storage medium according to the ninth embodiment of the present invention;

FIG. 13 shows an example embodiment of the system according to the tenth embodiment of the present invention; and

FIG. 14 shows a diagram relating to experiments with trained machine learning algorithms.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. At least one embodiment of the present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

At least one embodiment of the present invention determines anomalies in processes in an automated manner. To this end, at least one embodiment of the present invention provides computer-implemented methods for training a machine learning algorithm to detect anomalies in processes. Embodiments of the present invention are directed to a corresponding data processing system, a computer-implemented method, a data processing system and a system for detecting anomalies in processes. The subject matter of the respective claims contains developments and embodiments of the present invention.

According to a first embodiment of the present invention, a computer-implemented method is for training a machine learning algorithm to detect anomalies in processes comprising:

providing a training dataset of a process, comprising a first number N of training usage sequences, wherein the first number N is greater than one (N>1);

creating a predefined second number K of bootstrap datasets, wherein the second number K is greater than one (K>1), based upon the training dataset by randomly drawing a predefined third number M of usage sequences from the training dataset, wherein each draw is made from the first number N of training usage sequences; and

creating a number of process trees equal to the second number K by creating each process tree based upon one of the bootstrap datasets by way of a process mining algorithm.

According to a second embodiment of the present invention, a computer-implemented method for training a machine learning algorithm to detect anomalies in processes comprises:

providing a training dataset of a process, comprising a first number N of training usage sequences, wherein the first number N is greater than one; and

creating a second number K of process trees, wherein the second number K is greater than one (K>1), by creating each process tree based upon the training dataset by way of a process mining algorithm, wherein in each operator node of each process tree, one of the possible split operators is randomly selected.

According to a third embodiment of the present invention, a data processing system comprises at least one processor or electronic circuit for performing the method according to the first or second embodiment of the present invention.

According to a fourth embodiment of the present invention, a computer program product comprises commands which, on execution of the computer program product by a computer, cause the computer to perform the method according to the first or second embodiment of the present invention.

According to a fifth embodiment of the present invention, the computer program product according to the fourth embodiment of the present invention is stored on a computer-readable data storage medium.

According to a sixth embodiment of the present invention, a computer-implemented method for detecting anomalies in processes, in particular anomalies in processes by medical devices, comprises:

receiving a usage sequence of a process.

determining a prediction vector based upon the received usage sequence by way of a trained machine learning algorithm that has been trained by the method according to the first or second embodiment of the present invention, wherein the prediction vector comprises for each process tree of the trained MLA a value indicating whether the usage sequence fits the process tree concerned;

determining a normalized fitness value from the prediction vector and the second number K; and

classifying the process based upon the normalized fitness value.

According to a seventh embodiment of the present invention, a data processing system comprises at least one processor or electronic circuit for performing the method according to the sixth embodiment of the present invention.

According to an eighth embodiment of the present invention, a computer program product comprises commands which, on execution of the computer program product by a computer, cause the computer to perform the method according to the sixth embodiment of the present invention.

According to a ninth embodiment of the present invention, the computer program product according to the eighth embodiment of the present invention is stored on a computer-readable data storage medium.

According to a tenth embodiment of the present invention, a system for detecting anomalies in processes comprises a medical device and the data processing system according to the ninth embodiment of the present invention. The medical device is designed to log usage sequences relating to processes. The data processing system is communicatively connected to the medical device and designed to receive the usage sequences from the medical device.

The method according to the second embodiment of the present invention is an alternative to the method according to the first embodiment of the present invention. Both methods, however, can be combined with each other without restrictions. Thus the following statements apply in full to both of the methods.

The term “process” is understood to mean here a sequence of a plurality of activities or process steps aimed at achieving a predefined or desired result. The process may be a procedural or application process, a production or manufacturing process, and the like, and also a combination of one or more thereof. For example, the process may be a diagnostic or examination process, a therapeutic or treatment process, an assembly process, a distribution process, a measurement process, a test process, a maintenance process, and the like, and also a combination of one or more thereof.

Processes performed at devices, and in particular at medical devices, (cf. using the device) are logged by the device itself or a monitoring device (“observer”). The activities performed by the (medical) device during the implementation of a process are logged as a usage sequence. The logged usage sequence reflects the sequence of activities performed successively (sequentially) or simultaneously (in parallel). In particular, a usage sequence can be represented as a vector of activities.

Thus the usage sequences or the training usage sequences can each comprise a sequence of activities or process steps during use of a medical device for diagnosis and/or treatment of a patient. For example, a usage sequence of the process “CT of a cranium or brain” may comprise the following activities/process steps:

a) Position the cranium in the isocenter of the CT scanner;
b) Perform a topogram/localizer scan;
c) Adjust scan parameters of the CT scanner;
d) Perform a spiral scan;
e) Adjust scan parameters of the CT scanner;
f) Administer contrast agent;
g) Perform a spiral scan;
h) Adjust scan parameters of the CT scanner;
i) Perform a spiral scan;
j) Move the patient out.

For example, a usage sequence of the process “EKG exercise stress test” may comprise the following activities/process steps:

a) Attach the electrodes to the patient;
a) Attach the blood pressure cuff to the patient;
a) Attach the pulse oximeter to the patient;
d) Measure the resting heart rate;
e) Measure the resting blood pressure;
f) Measure the resting oxygen saturation;
g) Derive a resting EKG of 5 s;
h₁) Increase the resistance of the cycle ergometer by 10 Watts;
i₁) Measure the stress heart rate;
j₁) Measure the stress blood pressure;
k₁) Measure the stress oxygen saturation;
l₁) Derive a stress EKG of length 10 s;
h₂) Increase the resistance of the cycle ergometer by 10 Watts;
i₂) Measure the resting heart rate;
j₂) Measure the resting blood pressure;
k₂) Measure the resting oxygen saturation;
l₂) Derive a stress EKG of length 10 s;
h₃) Increase the resistance of the cycle ergometer by 10 Watts;
i₃) Measure the resting heart rate;
j₃) Measure the resting blood pressure;
k₃) Measure the resting oxygen saturation;
l₃) Derive a stress EKG of length 10 s;
. . .
hₙ) Increase the resistance of the cycle ergometer by 10 Watts;
iₙ) Measure the resting heart rate;
jₙ) Measure the resting blood pressure;
kₙ) Measure the resting oxygen saturation;
lₙ) Derive a resting EKG or stress EKG of length 10 s;
m) Wait without stress for a length of 60 s;
n) Measure the resting heart rate;
o) Measure the resting blood pressure;
p) Measure the resting oxygen saturation;
q) Remove the pulse oximeter from the patient;
r) Remove the blood pressure cuff from the patient;
s) Remove the electrodes from the patient.

A medical device, by which, or in which, processes performed at the medical device are logged as usage sequences, may be, for instance, an imaging device (such as an MRT device, CT device, PET device, SPECT device, PET-MRT device, PET-CT device, X-ray device, mammography device, etc.), a laboratory diagnostic device (e.g. automated urine analyzer, automated blood analyzer, automated immunoassay system, etc.) a lung function test device, an (exercise stress) EKG system, a gait analysis system, and the like.

Hence the process may be, for example, an imaging process (e.g. MRT of the head, CT of the knee, and the like), and the usage sequences may be sequences of activities that are, or were, performed in order to create the desired images.

The term “machine learning algorithm” (MLA) is understood to mean here a model created by training the machine learning algorithm. The trained machine learning algorithm according to the present invention, also referred to here as a “random process forest”, is intended to be able to classify as “normal” or “abnormal”, executions of the process on which the machine learning algorithm was trained. In this case, an execution, or the resultant usage sequence, is classified as “normal” if the execution corresponds to a specified standardized process or if the resultant usage sequence equates to a corresponding guideline protocol sequence. On the other hand, an execution, or the resultant usage sequence, is classified as “abnormal”, i.e. there is an anomaly, if the execution does not correspond to the specified standardized process or if the resultant usage sequence does not equate to the corresponding guideline protocol sequence.

In general, supervised learning, semi-supervised learning or unsupervised learning can be used in training a machine learning algorithm.

In the case of supervised learning, the training dataset comprises training data, each instance of which is provided with a label. The labels each indicate what is the correct result for the respective instances of training data (e.g. in the case of a classification task, the class to which the particular training-data instance belongs). During training of a machine learning algorithm based on supervised learning, the algorithm is thus adapted or trained based upon the difference between a result predicted by the algorithm and the correct result (label).

In the case of unsupervised learning, the training data of the training dataset has no labels. Thus unsupervised learning denotes machine learning without target values known a priori, and without reward from the environment. Unsupervised learning attempts to recognize patterns in the training data that differ from the structureless noise. Typical examples of unsupervised learning are automatic segmentation (clustering) or compression (of training data) for the purpose of dimension reduction. Creating a “random forest” from a plurality of uncorrelated decision trees based on training data is also a form of unsupervised learning. A trained random forest is a classifier that comprises a plurality of uncorrelated decision trees. All the decision trees are grown (have been created) under a certain type of randomization during the unsupervised learning process. For a classification, each decision tree of the trained random forest is allowed to make a decision, and the class having the most votes decides the final classification.

In the present context, the training dataset of the process comprises the first number N of training usage sequences. The training usage sequences may be stored usage sequences of N implementations of the process that is meant to be examined for anomalies. The first number N can equal preferably between 1000 and 100,000 (1000<=N<=100,000) and particularly preferably between 10,000 and 25,000 (10,000<=N<=25,000). For example, logged usage sequences of a medical device (e.g. MRT device) of a hospital or clinic can be used as the training dataset. Since unsupervised learning is being used, the usage sequences do not need to be provided (manually) with labels, and the machine learning algorithm can be trained using the unlabeled usage sequences as the training dataset.

In the method according to the first embodiment of the present invention, the predefined number K of bootstrap datasets is created, wherein K is greater than one (K>1). This is done by performing what is known as bootstrapping based upon the training dataset. For each of the bootstrap datasets, M training usage sequences are randomly drawn from the training dataset containing the N training usage sequences. In this process, each draw includes replacement, and therefore each random draw is performed from the (complete) first number N of training usage sequences. In other words, every time after one of the training usage sequences has been drawn randomly from the training dataset and has been saved in one of the bootstrap datasets, the drawn usage sequence is returned to the training dataset. Thus identical usage sequences can occur more than once in the bootstrap datasets. The third number M of randomly drawn usage sequences of the bootstrap datasets is greater than one (M>1).

The K process trees are then created based upon the K bootstrap datasets. This is done by creating, by way of the process mining algorithm, exactly one process tree based upon each of the bootstrap datasets. Possible process mining algorithms for creating the process trees are the Inductive Miner algorithm, the Alpha Miner algorithm, the Heuristic Miner algorithm, the Evolutionary Tree Miner algorithm, and the like.

A process tree is a compact and abstract representation of a block-structured workflow; in other words, a model of a process in the form of a tree. The process tree is a subtype of Petri nets which satisfies the criterion of soundness, and comprises places or activity nodes, also called leaves, containing activities or silent activities, and transitions or operator nodes, also called internal nodes, containing split operators. Activities may be observable actions, steps and measures, which may exist as logged usage sequences. Silent activities may be unobservable actions, steps and measures, which may be present implicitly in the usage sequences. Each operator node is provided with a split operator. The operator nodes thereby define by way of the split operators, the sequence in which the activities of the state nodes can, or must, be carried out. The following four types of split operators exist:
sequential composition (“sequence” or SEQ);
exclusive OR (XOR)
parallel composition (“parallel” or PAR);
loop (“redo” or LOOP).

The criterion for soundness is satisfied if the following criteria are met:
Safety: no place/activity node can hold more than one token at once.
Proper completion: after every run of the model, the model contains only one token at the end place/last activity node.
Option to complete: the model run can be completed from any state.
No dead sections: the model does not contain any transitions/operator nodes that can never be reached.

The random drawing of the usage sequences and the predefined number K of bootstrap datasets introduce randomness into the created process trees, i.e. the created process models. This ensures that, despite the limited number N of training usage sequences, as many different executions as possible of the process or of its usage sequences that can be considered normal are also identified or classified as normal by the trained machine learning algorithm.

In the method according to the second embodiment of the present invention, bootstrap datasets are not created, and the second number K of process trees is created directly from the training dataset. In order, nonetheless, to ensure randomness in the generated process trees, one of the possible split operators is randomly selected in each operator node when creating the process trees using the process mining algorithm. Instead of a deterministic selection of the split operators in each split node (for instance first check whether XOR possible, if so, select XOR, if not, check whether SEQ possible, if so, select SEQ, if not, check whether PAR possible, if so, select PAR, if not, check whether LOOP possible, if so, select LOOP), it is first ascertained for each operator node, which of the split operators are possible, and from these, one of the possible split operators is randomly selected.

The random selection of one of the possible split operators in each operator node of each of the K process trees introduces randomness into the created process trees, i.e. the created process models. This ensures that, despite the limited number N of training usage sequences, as many different executions as possible of the process or of its usage sequences that can be considered normal are also identified or classified as normal by the trained machine learning algorithm.

The pre-trained machine learning algorithm (random process forest), which has been trained, as described previously, to detect anomalies in processes, has K uncorrelated process trees, i.e. different process models. If a usage sequence is supplied as an input to the trained machine learning algorithm, the algorithm checks which of its process trees might have produced the supplied usage sequence; in other words, which process trees the supplied usage sequence fits. As an output, the trained machine learning algorithm produces a corresponding prediction vector, which comprises one binary value for each of the process trees, where a one (“1”) is entered in the prediction vector in the relevant position if a process tree fits the supplied usage sequence, and if not, a zero (“0”) is entered.

The usage sequences to be checked can be sent from the (medical) device directly to the data processing system on which the method for detecting anomalies in the process, or the trained machine learning algorithm (random process forest), is being performed. Alternatively, the usage sequences can first be sent to a central system (e.g. hospital management system), and forwarded from there to the data processing system for detecting anomalies in the process.

The normalized fitness value is calculated for the supplied usage sequence based upon the prediction vector and the second number K of process trees. This is done by summing the binary values of the prediction vector and then dividing (normalizing) by the second number K. The resultant normalized fitness value is a probability value, and indicates the likelihood that the checked usage sequence originates from a normal implementation of the process or the likelihood that the checked usage sequence originates from the process from the stored usage sequences of which the training dataset was created.

Based upon the normalized fitness value, it is possible to classify the process or the implementation thereof from which the usage sequence originates as “normal” or “abnormal”. If its normalized fitness value is greater than or equal to a predefined limit value, the process/execution thereof is classified as “normal”, and if its normalized fitness value is less than the predefined limit value, the process/execution thereof is classified as “abnormal”, and an anomaly is detected.

If a plurality of usage sequences are checked jointly (as a batch) by the trained machine learning algorithm (random process forest), then the resultant prediction vectors of each of the checked usage sequences are combined into a prediction matrix. In this process, the prediction vectors of the checked usage sequences can be entered as rows or columns of the prediction matrix. Then the prediction matrix is normalized (row by row or column by column) by the second number K to form a fitness vector. The fitness vector comprises a fitness value for each of the checked usage sequences.

The machine learning algorithm (random process forest) for detecting anomalies in a process can be created, or trained, according to the present invention based upon unlabeled training usage sequences based upon unsupervised learning. Hence there is no need for labor-intensive and time-consuming labeling of usage sequences in order to provide the training dataset.

According to a development of the present invention, the method according to the first or second embodiment of the present invention comprises:
Providing a training dataset of a process, comprising a first number N of training usage sequences, wherein the first number N is greater than one (N>1);
Creating a predefined second number K of bootstrap datasets, wherein the second number K is greater than one (K>1), based upon the training dataset by randomly drawing a predefined third number M of usage sequences from the training dataset, wherein each draw is made from the first number N of training usage sequences;
Creating a number of process trees equal to the second number K by creating each process tree based upon one of the bootstrap datasets by way of a process mining algorithm, wherein in each operator node of each process tree, one of the possible split operators is randomly selected.

During training of the machine learning algorithm, bootstrap datasets (according to the first embodiment of the present invention) are created, and also, in each operator node of each process tree, one of the possible split operators is randomly selected (according to the second embodiment of the present invention). This effectively introduces randomness at two points in the training of the machine learning algorithm.

By combining the two methods for training according to the present invention, the machine learning algorithm (random forest process) trained thereby can classify usage sequences as “normal or “abnormal” even more reliably, and hence identify anomalies in the process even more precisely.

According to a development of an embodiment of the present invention, the process mining algorithm is an Inductive Miner algorithm.

The Inductive Miner algorithm or Inductive Miner Directly-Follows based (IMDFb) algorithm creates process trees based upon (training) usage sequences. This is done by first creating a directly-follows graph based upon the different sequences of activities of the usage sequences of a dataset (training dataset or bootstrap dataset). The directly-follows graph reflects the dependencies between the different activities. An edge is inserted between two different activities if one of the two activities is followed directly by the other. The directly-follows graph is an essential step in the Inductive Miner algorithm for transforming the usage sequences into a process tree.

Each of the four split operators provides a specific transition feature which can be observed in the directly-follows graph. Repeatedly subdividing the directly-follows graph into partitions, i.e. into non-overlapping subdivisions of activities, is used to check whether one of the four split operators can link the activities. The XOR operator, for example, represents the appropriate link between a plurality of unconnected activities in the directly-follows graph, whereas the SEQ operator represents the appropriate link between a plurality of activities having directed connections therebetween in the directly-follows graph. On the other hand, the PAR operator represents the appropriate link for blocks that are connected to each other but also have a common output direction in the directly-follows graph. Similarly, the LOOP operator represents the appropriate link between a plurality of activities that are connected to each other in the directly-follows graph and have a specific start and end activity but for which no shortcuts are allowed. The process tree can hence be generated from the directly-follows graph.

The Inductive Miner algorithm allows the process trees to be created particularly efficiently and robustly from the training dataset or the bootstrap datasets.

According to a development of an embodiment of the present invention, the machine learning algorithm is a random forest algorithm comprising the second number K of process trees.

A random forest algorithm is created by way of the method for training a machine learning algorithm to detect anomalies in processes. The random forest algorithm comprises the second number K of process trees, each of which has been created based upon the training usage sequences of the training dataset or of the individual bootstrap datasets. The accordingly trained machine learning algorithm is called a “random process forest”.

According to a development of an embodiment of the present invention, the second number K lies in the range 50 to 200 (50<K<200). Preferably, the second number K equals 100 (K=100).

As it increases in value, the second number K of process trees created for the machine learning algorithm (random process forest) (and thus also the second number K of bootstrap datasets) converges towards an optimum. The computational effort likewise increases with an increasing second number K. From empirical trials, a second number K of 50 to 200, and in particular of 100, proved sufficient for a robust classification of the usage sequences for an at the same time reasonable computational effort.

According to a development of the method for training a machine learning algorithm to detect anomalies in processes, the third number M equals the first number N (M=N). Alternatively, the third number M is less than the first number N (M<N).

The third number M of usage sequences in the bootstrap datasets equals the first number N of usage sequences of the training dataset (M=N). Preferably, the third number M of usage sequences in the bootstrap datasets is less than the first number N of usage sequences of the training dataset (M<N) in particular if the first number N of usage sequences in the training dataset is very large (N>10,000). The third number M of usage sequences in the bootstrap datasets is always greater than 1, however.

Reducing the third number M of usage sequences in the bootstrap datasets can reduce significantly the computing effort and the time needed to train the machine learning algorithm (random process forest), while still allowing the trained machine learning algorithm to classify reliably usage sequences to be checked. Thus the computing effort and the training time for the machine learning algorithm falls if the third number M is less than the first number N.

According to a development of an embodiment of the present invention, the usage sequences are usage sequences of a medical device, in particular of a medical imaging device.

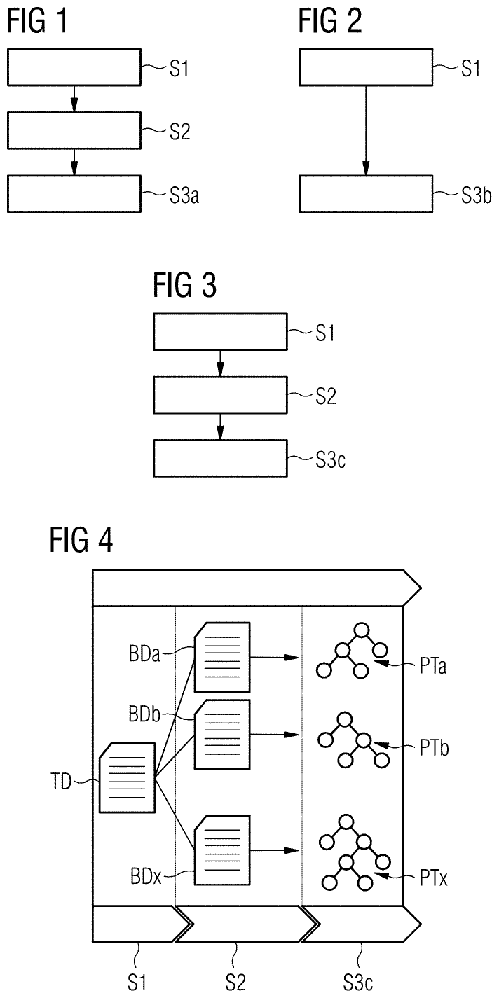

FIG. 1 shows schematically an example embodiment of the computer-implemented method for training a machine learning algorithm (MLA) to detect anomalies in processes according to the first embodiment of the present invention, and of the corresponding computer program product according to the fourth embodiment of the present invention. The method comprises the steps of providing S1 a training dataset TD, creating S2 a predefined second number K of bootstrap datasets BD, and creating S3a a number of process trees PT.

A training dataset TD is provided in the step of providing S1 a training dataset TD. The training dataset TD can be retrieved or forwarded from a memory, for instance a memory of a hospital management system. The training dataset TD comprises data from a process, for instance from a magnetic resonance tomography (MRT) examination of the head, in the form of usage sequences. The usage sequences represent implemented flows of activities that were carried out during implementation of the process. To this end, individual activities are stored in the form of a vector in the usage sequences in the order in which they were carried out. Thus each usage sequence reflects an (actually performed) implementation of the process. The training dataset comprises a first number N of training usage sequences of (actually performed) implementations of the process, wherein the first number N lies between 1,000 and 100,000 (1,000<=N<=100,000). For example, the training dataset TD can comprise 17,000 training usage sequences of (actually performed) implementations of the process (MRT examination of the head) (N=17,000).

A predefined second number K of bootstrap datasets BD is created in the step of creating S2 a predefined second number K of bootstrap datasets BD. The second number K equals the number of process trees PT that are subsequently meant to be created. The K bootstrap datasets BD are created successively by random drawing with replacement, also known as bootstrapping, from the training dataset TD containing the N training usage sequences. This is done by randomly drawing a third number M of training usage sequences from the training dataset TD for each bootstrap dataset BD, wherein each drawn training usage sequence is returned to the training dataset TD, and therefore the draw is each time made from the first number N of training usage sequences. Hence individual training usage sequences can occur more than once in the bootstrap datasets BD. The third number M of drawn training usage sequences in the bootstrap datasets BD is always greater than one and less than or equal to the first number N of training usage sequences of the training dataset TD (1<M<=N).

A number of process trees PT that is equal to the second number K is created in the step of creating S3a a number of process trees PT. The second number K of process trees PT or of bootstrap datasets BD is always greater than 1 (K>1) and preferably can be greater than or equal to 50 and less than or equal to 200 (50<=K<=200). For example, the second number K can equal 100 here (K=100). The second number K of process trees PT is created by a process mining algorithm, for instance in this case by the Inductive Miner algorithm, based upon the K bootstrap datasets BD. In this process, exactly one process tree PT is created from each of the K bootstrap datasets BD. The entirety of the created process trees PT results in the trained machine learning algorithm, called the random process forest, in which each process tree represents or models a possible flow of the process.

By creating the process trees PT of the machine learning algorithm (random process forest) from the bootstrap datasets BD, which have a certain randomness as a result of the random drawing with replacement (bootstrapping), a certain randomness also exists in the created process trees PT. Thus a certain randomness underlies the possible flows of the process that are modeled by the process trees PT. This counteracts the limited nature of the training dataset TD, which comprises only a limited number of different flows of the process (by the N training usage sequences) for training the machine learning algorithm (random process forest). There is also no need for time-consuming labeling of the usage sequences when preparing the training dataset TD, because training of the machine learning algorithm (random process forest) is unsupervised. Usage sequences of the process can be classified reliably and robustly as “normal” or “abnormal” using the trained machine learning algorithm (random process forest).

FIG. 2 shows schematically an example embodiment of the computer-implemented method for training a machine learning algorithm (MLA) to detect anomalies in processes according to the second embodiment of the present invention, and of the corresponding computer program product according to the fourth embodiment of the present invention. The method is an alternative to the method according to the first embodiment of the present invention and as shown in FIG. 1, and has some elements in common with the latter. Therefore only the differences from the method according to the first embodiment of the present invention and as shown in FIG. 1 are highlighted below. The method comprises the steps of providing S1 a training dataset TD and creating S3b a second number K of process trees PT.

The step of providing S1 a training dataset TD corresponds to that of the method according to the first embodiment of the present invention and as shown in FIG. 1.

Unlike the method according to the first embodiment of the present invention and as shown in FIG. 1, the method does not comprise a step of creating S2 a predefined second number K of bootstrap datasets BD, i.e. does not comprise bootstrapping the training dataset TD.

The second number K of process trees PT is created in the step of creating S3b a second number K of process trees PT. The second number K of process trees PT is always greater than 1 (K>1) and preferably can be greater than or equal to 50 and less than or equal to 200 (50<=K<=200). For example, the second number K can equal 100 here (K=100). The second number K of process trees PT is created by a process mining algorithm, for instance in this case by the Inductive Miner algorithm, based upon the training dataset TD. In this case, all the process trees PT are each created from the (one) training dataset TD. In addition, in each operator node of each process tree PT, one of the split operators possible in that particular case is randomly selected. Thus when each process tree PT is created, it is first ascertained for each of its operator nodes, which of the four split operators, XOR operator, SEQ operator, PAR operator and LOOP operator, are possible in the particular operator node, and then one of the split operators that are possible in the particular operator node is selected. The entirety of the created process trees PT results in the trained machine learning algorithm, called the random process forest, in which each process tree represents or models a possible flow of the process.

By creating the process trees PT of the machine learning algorithm (random process forest) by randomly selecting in each operator node of the process trees PT one of the split operators possible in that particular case, a certain randomness exists in the created process trees PT. Thus a certain randomness underlies the possible flows of the process that are modeled by the process trees PT. This counteracts the limited nature of the training dataset TD, which comprises only a limited number of different flows of the process (by the N training usage sequences) for training the machine learning algorithm (random process forest). There is also no need for time-consuming labeling of the usage sequences when preparing the training dataset TD, because training of the machine learning algorithm (random process forest) is unsupervised. Usage sequences of the process can be classified reliably and robustly as “normal” or “abnormal” using the trained machine learning algorithm (random process forest).

FIG. 3 shows schematically an example embodiment of a development of the computer-implemented method for training a machine learning algorithm (MLA) to detect anomalies in processes according to the first or second embodiment of the present invention, and of the corresponding computer program product according to the fourth embodiment of the present invention. The method is a combination of the methods according to the first and second embodiments of the present invention and as shown in FIGS. 1-2. Therefore only the differences from the method according to the first or second embodiment of the present invention and as shown in FIG. 1 or 2 respectively are highlighted below. The method comprises the steps of providing S1 a training dataset TD, creating S2 a predefined second number K of bootstrap datasets BD, and creating S3c a second number K of process trees PT.

The steps of providing S1 a training dataset TD and of determining a predefined second number K of bootstrap datasets BD correspond to those of the method according to the first embodiment of the present invention and as shown in FIG. 1.

A number of process trees PT that is equal to the second number K is created in the step of creating S3c a second number K of process trees PT, which step is a combination of the steps S3a and S3b. The second number K of process trees PT is created by a process mining algorithm, for instance in this case by the Inductive Miner algorithm, based upon the K bootstrap datasets BD. In this process, exactly one process tree PT is created from each of the K bootstrap datasets BD. In addition, in each operator node of each process tree PT, one of the split operators possible in that particular case is randomly selected. Thus when each process tree PT is created, it is first ascertained for each of its operator nodes, which of the four split operators, XOR operator, SEQ operator, PAR operator and LOOP operator, are possible in the particular operator node, and then one of the split operators that are possible in the particular operator node is selected. The entirety of the created process trees PT results in the trained machine learning algorithm, called the random process forest, in which each process tree represents or models a possible flow of the process.

By creating the process trees PT of the machine learning algorithm (random process forest) from the bootstrap datasets BD, which have a certain randomness as a result of the random drawing with replacement (bootstrapping), a certain randomness also exists in the created process trees PT. In addition, creating the process trees PT of the machine learning algorithm (random process forest) by randomly selecting in each operator node of the process trees PT, one of the split operators possible in that particular case, introduces an additional randomness into the created process trees PT. Thus a “combined” randomness underlies the possible flows of the process that are modeled by the process trees PT. This counteracts the limited nature of the training dataset TD, which comprises only a limited number of different flows of the process (by the N training usage sequences) for training the machine learning algorithm (random process forest). There is also no need for time-consuming labeling of the usage sequences when preparing the training dataset TD because training of the machine learning algorithm (random process forest) is unsupervised. Usage sequences of the process can be classified reliably and robustly as “normal” or “abnormal” using the trained machine learning algorithm (random process forest).

FIG. 4 shows again schematically the method from FIG. 3. In step S2, the K bootstrap datasets BD, each comprising M usage sequences, are created by random drawing with replacement (bootstrapping) from the (one) training dataset TD containing the N training usage sequences that was provided in step S1. For example in this case, the third number M can equal the first number N. Then in step S3c, exactly one process tree PTa, PTb, . . . , PTx of the random process forest is created from each of the K bootstrap datasets BD by way of a process mining algorithm, in particular by way of the Inductive Miner algorithm. Each of the uncorrelated K process trees PTa, PTb, . . . , PTx of the random process forest models a possible flow, i.e. K different flows, of the process, each having a certain randomness in the flow. Thus the random process forest trained by unsupervised learning comprises K uncorrelated process trees PTa, PTb, . . . , PTx, which have a certain “combined” randomness by virtue of the previously described bootstrapping and random selection of possible split operators, thereby counteracting the limited nature of the training dataset TD. Usage sequences of the process can therefore be classified reliably and robustly as “normal” or “abnormal” using the trained random process forest.

FIG. 5 shows schematically a directly-follows graph DFG, which has been created from a dataset (the training dataset TD or one of the bootstrap datasets BD) by a first step of the Inductive Miner directly-follows algorithm.

In this case, the dataset TD, BD comprises the four (N=4) usage sequences {three times <a,b,d,e>; <a,d,b,e>; <a,d,c,e>; <a,c,d,e>} by way of example. Each of the usage sequences comprises a sequence of activities a, b, c, d and e in vector form. The directly-follows graph DFG is created from the dataset TD, BD. The activity a is always followed by a combination of the activities b, c and d, and subsequently by the activity e. In addition, frequencies can be specified as a numerical value on the connections (edges) between the activities in the directly-follows graph DFG.

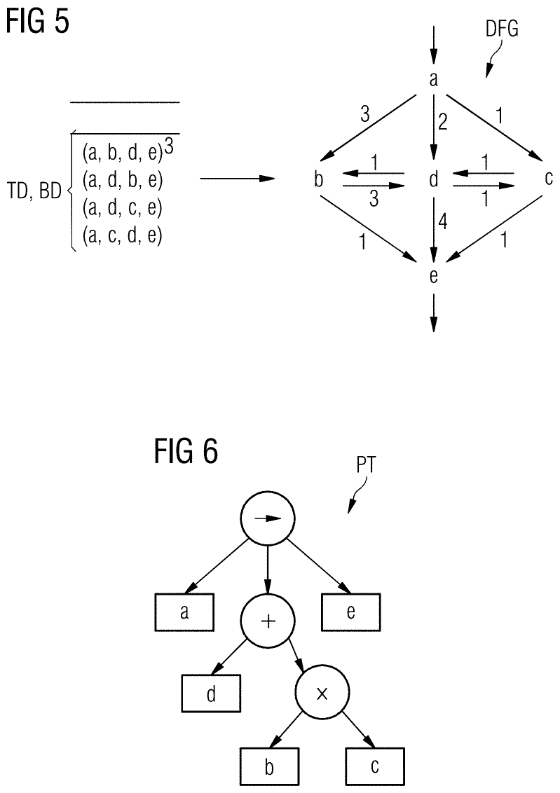

FIG. 6 shows schematically a process tree PT, which has been created from a directly-follows graph DFG, in this case the directly-follows graph DFG of FIG. 5, by a second step of the Inductive Miner algorithm.

The activities b, c, and d of the dataset TD, BD, as shown in the directly-follows graph DFG, can follow the activity a only sequentially. Likewise, the activity e can only follow the activities b, c, and d sequentially. Thus the activity node containing the activity a is connected to the activity node containing the activity e via the operator node containing the split operator SEQ (→). Since the combinations of the activities b, c and d run between the sequential activities a and e, the operator node containing the split operator SEQ (→) is connected to the operator node containing the split operator PAR (+), which connects the activity node containing the activity d to the operator node that connects the two activity nodes containing the activities b and c. The activity node containing the activity d is connected via the operator node containing the split operator PAR (+) to the operator node that connects the two activity nodes containing the activities b and c, because the activity d can both precede and succeed both the activity b and the activity c, and thus runs in parallel to these activities. The activity node containing the activity b is connected to the activity node containing the activity c via the operator node containing the split operator XOR (x), because either the activity b or the activity c precedes or succeeds the activity d, i.e. runs in parallel thereto.

If a plurality of the four split operators are possible in an operator node, in the conventional Inductive Miner algorithm (e.g. in the method according to the first embodiment of the present invention) the first possible split operator, according to the specified order XOR, SEQ, PAR, LOOP, is assigned to the particular operator node concerned (“greedy”). In the modified Inductive Miner algorithm according to the second embodiment of the present invention, one of the split operators possible in an operator node is randomly assigned (for instance if XOR or PAR were possible in an operator node, one of the two possible split operators is selected randomly).

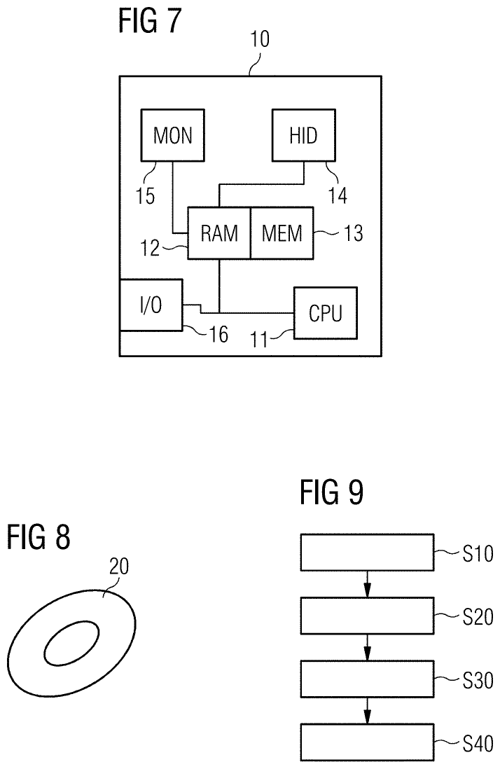

FIG. 7 shows schematically an example embodiment of the data processing system 10 according to the third embodiment of the present invention. The data processing system 10 can perform the step S1, the step S2 and one of the steps S3a, S3b or S3c of the methods according to the first or second embodiment of the present invention and as shown in FIGS. 1-3.

The data processing system 10 may be a personal computer (PC), a laptop, a tablet, a server, a distributed system (e.g. cloud system), and the like. The data processing system 10 comprises a central processing unit (CPU) 11, a memory, which has a random access memory (RAM) 12 and a non-volatile memory (MEM, e.g. hard disk) 13, a human interface device (HID, e.g. keyboard, mouse, touchscreen, etc.) 14, an output device (MON, e.g. monitor, printer, loudspeaker, etc.) 15, and an interface (input/output, I/O, e.g. USB, Bluetooth, WLAN, etc.) 16 for receiving and sending data. The CPU 11, the RAM 12, the HID 14, the MON 15 and the I/O 16 are communicatively connected via a databus. The RAM 12 and the MEM 13 are communicatively connected via another databus.

The computer program product according to the fourth embodiment of the present invention, which can be stored on the computer-readable medium 20 (see FIG. 8), can be stored in the MEM 13 and loaded from there or from the computer-readable medium 20 into the RAM 12. In accordance with the computer program product, the CPU 11 performs the step S1, the step S2 and one of the steps S3a, S3b or S3c. A user (staff member of the rescue coordination center) can initialize and control the execution via the HID 14. The status and the result of the executed computer program can be displayed by the MON 15 or forwarded via the I/O 16 to the user. The result of the executed computer program can be stored permanently on the non-volatile memory MEM 13 or another computer-readable medium.

In particular, the CPU 11 and the RAM 12 for executing the computer program can comprise a plurality of CPUs 11 and a plurality of RAMs 12, for example in a computer cluster or a cloud system. The HID 14 and the MON 15 for controlling the execution of the computer program can be comprised by another data processing system such as a terminal, which is communicatively connected to the data processing system 10 (e.g. cloud system).

FIG. 8 shows schematically an example embodiment of the computer-readable data storage medium 20. In this case, the computer program product according to the fourth embodiment of the present invention is stored by way of example on a computer-readable memory disk 20 such as a compact disc (CD), digital video disc (DVD), high definition DVD (HD DVD) or Blu-ray disc (BD), the computer program product comprising instructions which, when executed by a data processing system (computer), cause the data processing system to perform the step S1, the step S2 and one of the steps S3a, s3b or s3c.

It is also possible, however, that the computer-readable data storage medium 20 is a data storage device such as a magnetic storage device (e.g. magnetic core memory, magnetic tape, magnetic card, magnetic strip, magnetic bubble memory, storage roll, hard disk, floppy disk or removable storage device), an optical storage device (e.g. holographic memory, optical tape, tesa tape, laser disc, Phasewriter (Phasewriter Dual, PD) or Ultra Density Optical (UDO)), a magneto-optical storage device (e.g. miniDisc or magneto-optical disk (MO disk)), a volatile semiconductor memory/solid-state memory (e.g. random access memory (RAM), dynamic RAM (DRAM) or static RAM (SRAM)) or a non-volatile semiconductor memory/solid-state memory (e.g. read-only memory (ROM), programmable ROM (PROM), erasable PROM (EPROM), electrically erasable PROM (EEPROM), flash EEPROM (e.g. USB stick), ferroelectric RAM (FRAM), magnetoresistive RAM (MRAM) or phase-change RAM).

FIG. 9 shows schematically an example embodiment of the computer-implemented method for detecting anomalies in processes, in particular anomalies in processes by medical devices, according to the sixth embodiment of the present invention. The method comprises the steps of receiving S10 a usage sequence, determining S20 a prediction vector PV, determining S30 a normalized fitness value FVAL, and classifying S40 the process.

A usage sequence of a process or of an implementation of the process is received in the step of receiving S10 a usage sequence. It is also possible for a plurality of usage sequences of a plurality of processes/process implementations to be received.

In the step of determining S20 a prediction vector PV, a prediction vector PV or a prediction matrix PM is determined based upon the received usage sequence(s) by way of the trained machine learning algorithm (random process forest), which was trained by the method according to the first or second embodiment and as shown in FIG. 1-3. The determined prediction vector PV or the determined prediction matrix PM comprises for each process tree PT of the trained machine learning algorithm (random process forest) a value that indicates whether the usage sequence, or the particular usage sequence of the usage sequences, fits the process tree PT concerned. Thus, if the received usage sequence/the particular usage sequence of the received usage sequences can originate from the process modeled by the process tree PT concerned, the corresponding value of the prediction vector PV/prediction matrix PM is set to one (“1”), and if not is set to zero (“0”).

In the step of determining S30 a normalized fitness value FVAL, a normalized fitness value FVAL or a normalized fitness vector FV is determined from the determined prediction vector PV or the determined prediction matrix PM respectively and from the second number K. This is done by summing the individual values of the prediction vector PV or prediction matrix PM that are associated with the received usage sequence/the particular usage sequence of the received usage sequences, and dividing by the second number K of process trees PT. The normalized fitness value FVAL or each of the normalized fitness values of the normalized fitness vector PV equals a probability. This probability indicates the likelihood that the corresponding usage sequence originates from the process, where the value one (FVAL=1) corresponds to a probability of 100% [percent] and the value zero (FVAL=0) corresponds to a probability of 0%.

In the step of classifying S40 the process, the process or the process implementation is classified based upon the normalized fitness value FVAL. The process or the implementation thereof can be classified as “normal” if its normalized fitness value FVAL is greater than or equal to 0.5 (FVAL>=0.5), preferably greater than or equal to 0.66 (FVAL>=0.66), particularly preferably greater than or equal to 0.75 (FVAL>=0.75). Otherwise the process or the implementation thereof is classified as “abnormal” and hence an anomaly is detected.

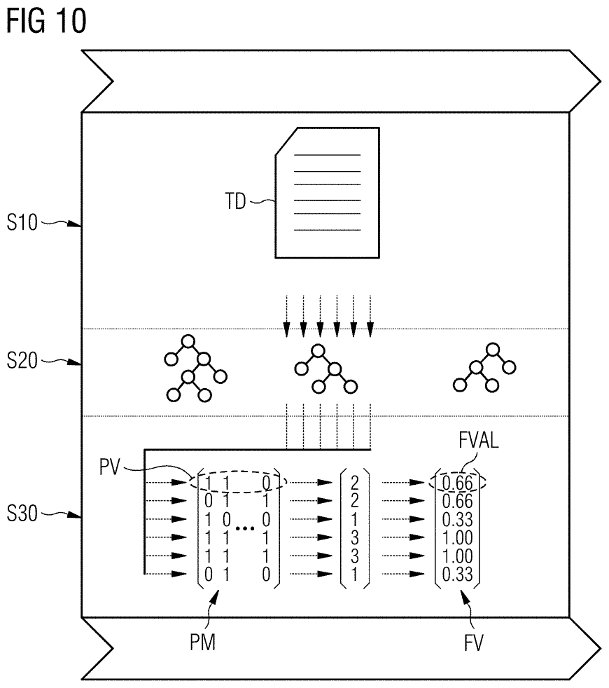

FIG. 10 shows schematically the method of FIG. 9 once again. From a plurality of usage sequences received in step S10 from a plurality of executions of the process, in step S20, a corresponding prediction vector PV is determined for each of the received usage sequences in the form of the prediction matrix PM by way of the trained random process forest. The individual values of the respective prediction vectors PV, which indicate whether the associated usage sequence fits the particular process tree of the K process trees PT of the trained random process forest, are summed for each usage sequence. Then, in step S30, the summed values are divided by the second number K of process trees PT of the trained random process forest in order to obtain the associated fitness value FVAL of the usage sequences, wherein the individual fitness values FVAL of the usage sequences are combined in the fitness vector FV. Based upon the individual fitness values FVAL, which indicate a probability that the associated usage data originates from the process, then in step S40, which is not shown here, the particular process or the particular implementation thereof can be classified as “normal” or “abnormal”.

FIG. 11 shows schematically an example embodiment of the data processing system 30 according to the seventh embodiment of the present invention. The data processing system 30 can perform the step S10, the step S20, the step S30 and the step S40 of the method according to the sixth embodiment of the present invention and as shown in FIG. 10.

The data processing system 30 may be a personal computer (PC), a laptop, a tablet, a server, a distributed system (e.g. cloud system), and the like. The data processing system 30 comprises a central processing unit (CPU) 31, a memory, which has a random access memory (RAM) 32 and a non-volatile memory (MEM, e.g. hard disk) 33, a human interface device (HID, e.g. keyboard, mouse, touchscreen, etc.) 34, an output device (MON, e.g. monitor, printer, loudspeaker, etc.) 35, and an interface (input/output, I/O, e.g. USB, Bluetooth, WLAN, etc.) 36 for receiving and sending data. The CPU 31, the RAM 32, the HID 34, the MON 35 and the I/O 36 are communicatively connected via a databus. The RAM 32 and the MEM 33 are communicatively connected via another databus.

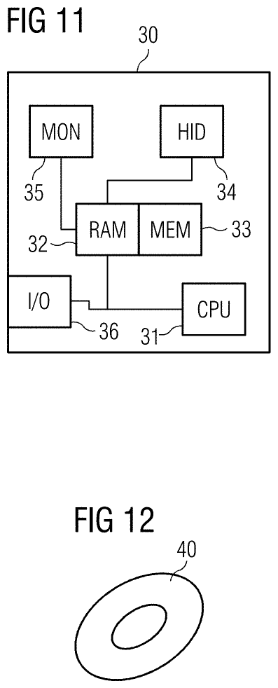

The computer program product according to the eighth embodiment of the present invention, which can be stored on the computer-readable medium 40 (see FIG. 12), can be stored in the MEM 33 and loaded from there or from the computer-readable medium 40 into the RAM 32. In accordance with the computer program product, the CPU 31 executes the step S10, the step S20, the step S30 and the step S40. A user (staff member of the rescue coordination center) can initialize and control the execution via the HID 34. The status and the result of the executed computer program can be displayed by the MON 35 or forwarded via the I/O 36 to the user. The result of the executed computer program can be stored permanently on the non-volatile memory MEM 33 or another computer-readable medium.

In particular, the CPU 31 and the RAM 32 for executing the computer program can comprise a plurality of CPUs 31 and a plurality of RAMs 32, for example in a computer cluster or a cloud system. The HID 34 and the MON 35 for controlling the execution of the computer program can be comprised by another data processing system such as a terminal, which is communicatively connected to the data processing system 30 (e.g. cloud system).

FIG. 12 shows schematically an example embodiment of the computer-readable data storage medium 40. In this case, the computer program product according to the eighth embodiment of the present invention is stored by way of example on a computer-readable memory disk 40 such as a compact disc (CD), digital video disc (DVD), high definition DVD (HD DVD) or Blu-ray disc (BD), the computer program product comprising instructions which, when executed by a data processing system (computer), cause the data processing system to perform step the S10, the step S20, the step S30 and the step S40.

It is also possible, however, that the computer-readable data storage medium 40 is a data storage device such as a magnetic storage device (e.g. magnetic core memory, magnetic tape, magnetic card, magnetic strip, magnetic bubble memory, storage roll, hard disk, floppy disk or removable storage device), an optical storage device (e.g. holographic memory, optical tape, tesa tape, laser disc, Phasewriter (Phasewriter Dual, PD) or Ultra Density Optical (UDO)), a magneto-optical storage device (e. g. miniDisc or magneto-optical disk (MO disk)), a volatile semiconductor memory/solid-state memory (e.g. random access memory (RAM), dynamic RAM (DRAM) or static RAM (SRAM)) or a non-volatile semiconductor memory/solid-state memory (e.g. read-only memory (ROM), programmable ROM (PROM), erasable PROM (EPROM), electrically erasable PROM (EEPROM), flash EEPROM (e.g. USB stick), ferroelectric RAM (FRAM), magnetoresistive RAM (MRAM) or phase-change RAM).

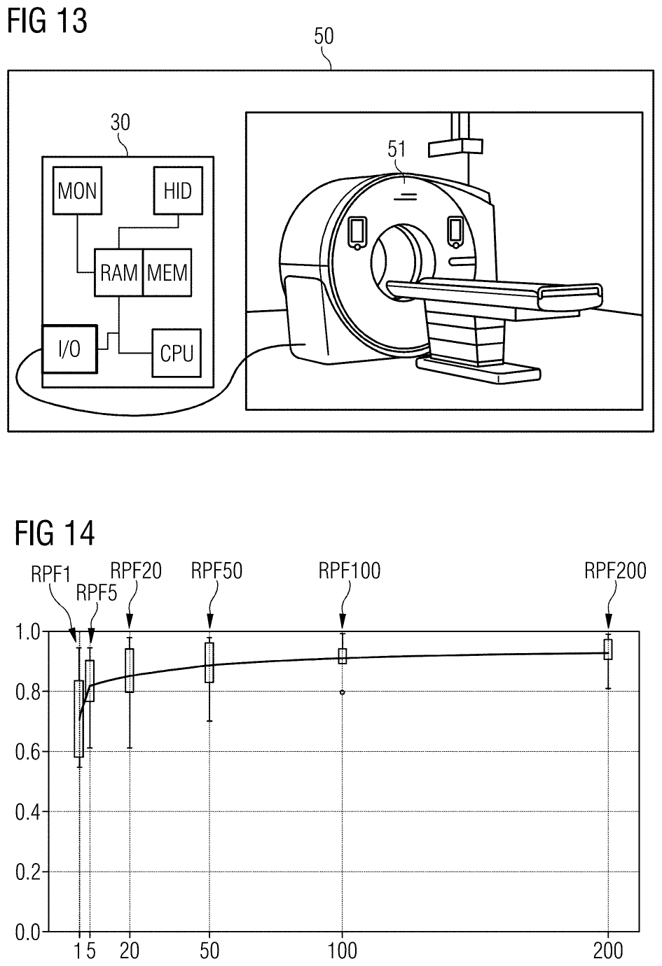

FIG. 13 shows schematically an example embodiment of the system 50 for detecting anomalies in processes according to the tenth embodiment of the present invention. The system 50 comprises a medical device 51 and the data processing system 30 according to the seventh embodiment of the present invention and as shown in FIG. 11.

The medical device 51 may be a medical imaging device, in particular an MRT device. The medical device 51 is designed to log usage sequences relating to processes or to implementations thereof.

The data processing system 30 is communicatively connected by way of the I/O 36 to the medical device 51 or to a controller of the medical device 51 (possibly indirectly via a central system such as a hospital management system).

The data processing system 30 receives (indirectly) the usage sequences from the medical device 51, and, in order to detect anomalies, performs the method according to the sixth embodiment of the present invention and as shown in FIG. 10.

FIG. 14 shows a diagram relating to experiments with trained machine learning algorithms. Six machine learning algorithms (random process forests) RPF1, RPF5, RPF20, RPF50, RPF100 and RPF200 have been trained, where RPF1 has a second number K of 1 (K=1) process trees PT, RPF5 has a second number K of 5 (K=5) process trees PT, RPF20 has a second number K of 20 (K=20) process trees PT, RPF50 has a second number K of 50 (K=50) process trees PT, RPF100 has a second number K of 100 (K=100) process trees PT, and RPF200 has a second number K of 200 (K=200) process trees PT. All six machine learning algorithms (random process forests) have been trained using the same training dataset TD of a process under consideration (e.g. MRT examination of the knee) according to the method of FIGS. 3 and 4.

The second number K of process trees (ensemble size) is plotted as the abscissa, and the performance of the six machine learning algorithms (random process forests) is plotted as the ordinate. It emerges that the performance of RPF50, RPF100 and RPF200 are close to one another and thus a second number K between 50 and 200 (50<=K<=200) is preferable. It also emerges that the performance of RPF100 in classifying usage sequences of implementations of the process is only slightly worse than that of RPF200. Since, however, the computing effort and thus the time taken both for training and for classification is significantly higher using RPF200 than with RPF100, the second number K equal to 100 (K=100) is particularly preferable.

Although specific embodiments have been illustrated and described here, it is obvious to a person skilled in the art that there are a multiplicity of alternatives and/or equivalent implementations. It must be recognized that the example designs or embodiments are just examples and are not intended to have any limiting effect whatsoever on the scope, applicability or configuration. In fact, the above summary and detailed description provides a person skilled in the art with sufficient prompting to implement at least one preferred embodiment, where it goes without saying that various alterations in the function and arrangement of the elements described in an example embodiment do not depart from the scope of application presented in the accompanying claims and the legal equivalents thereof. This application is generally intended to cover all adaptations or variations of the specific embodiments discussed here.

In the above detailed description, various features have been combined in one or more examples in order to keep the disclosure brief. It is self-evident that the above description is intended to be illustrative and not restrictive. It is intended to cover all the alternatives, modifications and equivalents that may be included in the invention. Numerous other examples will become obvious to a person skilled in the art on studying the above disclosure.

In order to facilitate a thorough understanding of the invention, a specific nomenclature is used, which has been used in the above disclosure. In the light of the specification contained therein, however, it will be obvious to a person skilled in the art that the specific details are not needed in order to apply the invention. Thus the above descriptions of specific embodiments of the present invention are presented for the purpose of illustration and explanation. They are not intended to be exhaustive or to restrict the invention to the precise embodiments disclosed above; obviously numerous modifications and variations regarding the aforementioned teaching are possible. The embodiments have been selected and described in order to best explain the principles of the invention and its practical applications, and hence to give other professionals the opportunity to apply in the best way the invention and different embodiments using various modifications as seems fit for the particular use. The terms “including” and “in which” are used throughout the specification as equivalents for the respective terms “comprising” and “wherein”. In addition, the terms “first”, “second”, “third”, etc. are used merely as a designation and are not intended to place numerical requirements on the objects or to define a specific order of precedence. In the context of the present description and the claims, the conjunction “or” shall be understood to be inclusive (“and/or”) and not exclusive (“either . . . or”).

Of course, the embodiments of the method according to the invention and the imaging apparatus according to the invention described here should be understood as being example. Therefore, individual embodiments may be expanded by features of other embodiments. In particular, the sequence of the method steps of the method according to the invention should be understood as being example. The individual steps can also be performed in a different order or overlap partially or completely in terms of time.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for training a machine learning algorithm to detect at least one anomaly in at least one process, comprising:
providing a training dataset of a process, including a first number of training usage sequences, the first number being greater than one;
creating a second number of bootstrap datasets, the second number being greater than one, based upon the training dataset by randomly drawing a third number of usage sequences from the training dataset, each draw of the drawing of the third number being made from the first number of training usage sequences; and
creating a number of process trees equal to the second number by creating each process tree, of the number of process trees, based upon one bootstrap dataset of the bootstrap datasets, using a process mining algorithm.

2. A computer-implemented method for training a machine learning algorithm to detect at least one anomaly in at least one process, comprising:
providing a training dataset of a process, including a first number of training usage sequences, the first number being greater than one; and
creating a second number of process trees, the second number being greater than one, by creating each process tree, of the number of process trees, based upon the training dataset using a process mining algorithm, wherein in each operator node of each process tree, one of a possible split of operators is randomly selected.

3. A computer-implemented method for training a machine learning algorithm to detect at least one anomaly in at least one process, comprising:
providing a training dataset of a process, including a first number of training usage sequences, the first number being greater than one;
creating a second number of bootstrap datasets, the second number being greater than one, based upon the training dataset by randomly drawing a third number of usage sequences from the training dataset, wherein each draw of the drawing of the third number is made from the first number of training usage sequences; and
creating a number of process trees equal to the second number by creating each process tree, of the number of process trees, based upon one of the bootstrap datasets using a process mining algorithm, wherein in each operator node of each process tree, one of a possible split of operators is randomly selected.

4. The computer-implemented method of claim 1, wherein the process mining algorithm is an Inductive Miner algorithm.

5. The computer-implemented method of claim 1, wherein the training usage sequences each include a sequence of activities during use of a medical device for at least one of diagnosis of a patient and treatment of the patient.

6. The computer-implemented method of claim 1, wherein the machine learning algorithm is a random forest algorithm comprising the second number of process trees.

7. The computer-implemented method of claim 1, wherein the second number lies in the range 50 to 200.

8. The computer-implemented method of claim 1, wherein the third number equals the first number, or alternatively wherein the third number is less than the first number.

9. The computer-implemented method of claim 1, wherein the usage sequences are usage sequences of a medical device.

10. A data processing system, comprising at least one processor or electronic circuit for performing at least the computer-implemented method of claim 1.

11. A computer-implemented method for detecting at least one anomaly in at least one process, comprising:
receiving a usage sequence of a process;
determining a prediction vector based upon the usage sequence received by use of a trained machine learning algorithm trained by the computer-implemented method of claim 1, wherein the prediction vector includes, for each respective process tree of the trained machine learning algorithm, a value indicating whether the usage sequence fits the respective process tree;
determining a normalized fitness value from the prediction vector and the second number; and
classifying the process based upon the normalized fitness value.

12. A system for detecting at least one anomaly in at least one process, comprising:
a medical device, embodied to log usage sequences relating to processes; and
the data processing system of claim 10, the data processing system being communicatively connected to the medical device and being embodied to receive the usage sequences from the medical device.

13. The computer-implemented method of claim 2, wherein the process mining algorithm is an Inductive Miner algorithm.

14. The computer-implemented method of claim 2, wherein the training usage sequences each include a sequence of activities during use of a medical device for at least one of diagnosis of a patient and treatment of the patient.

15. The computer-implemented method of claim 2, wherein the machine learning algorithm is a random forest algorithm comprising the second number of process trees.

16. The computer-implemented method of claim 3, wherein the process mining algorithm is an Inductive Miner algorithm.

17. The computer-implemented method of claim 3, wherein the training usage sequences each include a sequence of activities during use of a medical device for at least one of diagnosis of a patient and treatment of the patient.

18. The computer-implemented method of claim 3, wherein the machine learning algorithm is a random forest algorithm comprising the second number of process trees.

19. The computer-implemented method of claim 4, wherein the process mining algorithm is an Inductive Miner algorithm.

20. The computer-implemented method of claim 4, wherein the training usage sequences each include a sequence of activities during use of a medical device for at least one of diagnosis of a patient and treatment of the patient.

21. The computer-implemented method of claim 4, wherein the machine learning algorithm is a random forest algorithm comprising the second number of process trees.

22. A data processing system, comprising at least one processor or electronic circuit for performing at least the computer-implemented method of claim 2.

23. A data processing system, comprising at least one processor or electronic circuit for performing at least the computer-implemented method of claim 3.

24. The computer-implemented method of claim 11, wherein the computer-implemented method is for detecting at least one anomaly in at least one process by at least one medical device.

25. A computer-implemented method for detecting at least one anomaly in at least one process, comprising:
receiving a usage sequence of a process;
determining a prediction vector based upon the usage sequence received by use of a trained machine learning algorithm trained by the computer-implemented method of claim 2, wherein the prediction vector includes, for each respective process tree of the trained machine learning algorithm, a value indicating whether the usage sequence fits the respective process tree;
determining a normalized fitness value from the prediction vector and the second number; and
classifying the process based upon the normalized fitness value.

26. A computer-implemented method for detecting at least one anomaly in at least one process, comprising:
receiving a usage sequence of a process;
determining a prediction vector based upon the usage sequence received by use of a trained machine learning algorithm trained by the computer-implemented method of claim 3, wherein the prediction vector includes, for each respective process tree of the trained machine learning algorithm, a value indicating whether the usage sequence fits the respective process tree;
determining a normalized fitness value from the prediction vector and the second number; and
classifying the process based upon the normalized fitness value.

27. A system for detecting at least one anomaly in at least one process, comprising:
a medical device, embodied to log usage sequences relating to processes; and
the data processing system of claim 22, the data processing system being communicatively connected to the medical device and being embodied to receive the usage sequences from the medical device.

28. A system for detecting at least one anomaly in at least one process, comprising:
a medical device, embodied to log usage sequences relating to processes; and
the data processing system of claim 23, the data processing system being communicatively connected to the medical device and being embodied to receive the usage sequences from the medical device.
